(19) United States
(12) Patent Application Publication
(10) Pub. No.: US 2021/0367636 A1
(43) Pub. Date: Nov. 25, 2021

(54) WIRELESS CHARGING OF MOBILE COMMUNICATION DEVICES

(71) Applicant: TRUST TECHNOLOGY WORLD DMCC, DUBAI (AE)

(72) Inventors: Bruce Holley, Honington (GB); Joshua Mihill, Grantham (GB)

(21) Appl. No.: 16/976,383

(22) PCT Filed: Feb. 27, 2019
(86) PCT No.: PCT/IB2019/051579

§ 371 Date: Aug. 27, 2020

(30) Foreign Application Priority Data
Feb. 27, 2018 (GB) 1803198.9

(51) Int. Cl. H04B 1/3827; G01R 29/08

(52) U.S. Cl. CPC H04B 1/3838; H02J 50/10; G01R 29/0857

(57) ABSTRACT

A communication device that emits radio frequency signals having a module to determine when the radio frequency signals may be present and are potentially harmful, and to activate a remedial signal generator if the radio frequency signals are considered to be potentially harmful; and the power to operate the module is from a power source that can be charged using wireless charging techniques.

### FIELD

The present teachings relate to a personal communication device that generates a remedial signal when the device emits potentially harmful radiation by a means including wireless charging.

### BACKGROUND

The present invention relates to the provision of remedial signals to compensate for potentially harmful radiation that may be emitted by handheld electronic devices. The fact that devices such as mobile telephones may emit potentially harmful radiation has been known and explored for many years. It has further been proposed that the potential harm that could be caused by such radiation can be reduced or eliminated by the superimposition of a remedial signal on the potentially harmful radiation. Examples of such proposals may be found in PCT Publications WO2015/124744 and WO2015/124689 which include the possibility of providing means for the detection of the radiation, the analysis of the radiation to determine if it is potentially harmful and to determine the nature of the radiation.

Additionally, the communication device may include means to activate a remedial signal according to the nature and strength of the radiation detected. Such proposals are particularly aimed at reducing the potential for harm to humans through use of mobile communication devices such as cellphones. The provision of the remedial signal can however cause a significant drain on the power of the device which is why WO2015/053528 describes a method to reduce battery consumption through effective power management while continuing to monitor RF radiation.

Effective power management and regular charging of portable battery powered devices are necessary. These types of devices and in particular portable communications devices can benefit from wireless charging technologies allowing them to be charged without a physical connection.

However, there is a continuing need to simplify the technology and to economise on space and weight within the mobile communication device whilst retaining and enhancing the ability to detect and analyse the radiation generated and to provide a remedial signal.

### SUMMARY

The present invention therefore provides a mobile communication device that emits radio frequency signals provided with a module to sense if the radio frequency signals are potentially harmful and to activate a remedial signal generator if the radio frequency is considered to be potentially harmful wherein the power to operate the module can be provided by wireless charging.

This invention is ideally suited to be used as part of or in combination with devices that emit potentially harmful radiation in close proximity to humans or animals particularly close to the head and which are electrically powered and require recharging.

Wireless charging is well known and provides a simple, convenient, and safe method of charging of electrical devices at home, work and also in industry environments. Wireless charging eliminates the need for a physical connector and cables and provides a number of advantages which are particularly relevant to electronic devices with rechargeable batteries that are used regularly and require regularly recharging. Wireless charging uses the principles of inductive power transfer or magnetic resonance and facilitates the transfer of electrical power from one point to another without any electrical contacts. It uses electromagnetic fields to safely transfer power from a transmitting device to a receiving device in order to charge or recharge a power source such as a battery. The transmitter and receiver comprise conductive materials such as copper coils or a copper trace on a printed circuit board.

Wireless charging is used extensively in many sectors and for both industrial devices and consumer devices. Typical advantages of wireless charging include reduced costs of maintaining mechanical connectors, safe charging of devices that are required to remain sterile, or designed to be waterproof, or must be charged in hazardous environments whereby sparks must be prevented.

Wireless communications devices are used regularly where increasing power demands are driven by rapidly increasing functionality which results in the need for regular charging. Consequently, they are ideally suited to take advantage of the benefits of wireless charging. Wireless charging capabilities are now being integrated directly into wireless communication devices such as cellular telephones. The wireless charging is preferably achieved by the provision of a conductive structure within the mobile communication device for receipt of transmission from a remote wireless charging transmitter. However, as indicated cellular telephones also emit potentially harmful radiation the effects of which can be reduced or eliminated through the superposition of a remedial signal and this signal can be generated by power derived from the wireless charging.

### BRIEF DESCRIPTION OF THE DRAWINGS

FIG. 1 illustrates a schematic of the communication device.

FIG. 2 illustrates a wireless charging assembly.

### DETAILED DESCRIPTION

In a preferred embodiment the mobile communication device is a portable telephone and a module to include the receiver of wireless charging is provided within the handset or the case of the portable telephone. The module itself may perform several functions and may itself comprise several modules. For example the overall control module for the delivery of a remedial signal by the portable telephone may consist of a detection module that senses the radiation emitted by the portable telephone, an analysis module that analyses the signal that is detected to determine if it is potentially harmful and also the nature of the radiation in order to determine the type, strength and/or duration of remedial signal that is appropriate for the radiation that has been sensed. The control module may also contain a remedial signal generation module which can be activated by the analysis module to generate the appropriate remedial signal. All these functions may be provided by a single microprocessor which is powered by a wirelessly charged power source such as the battery.

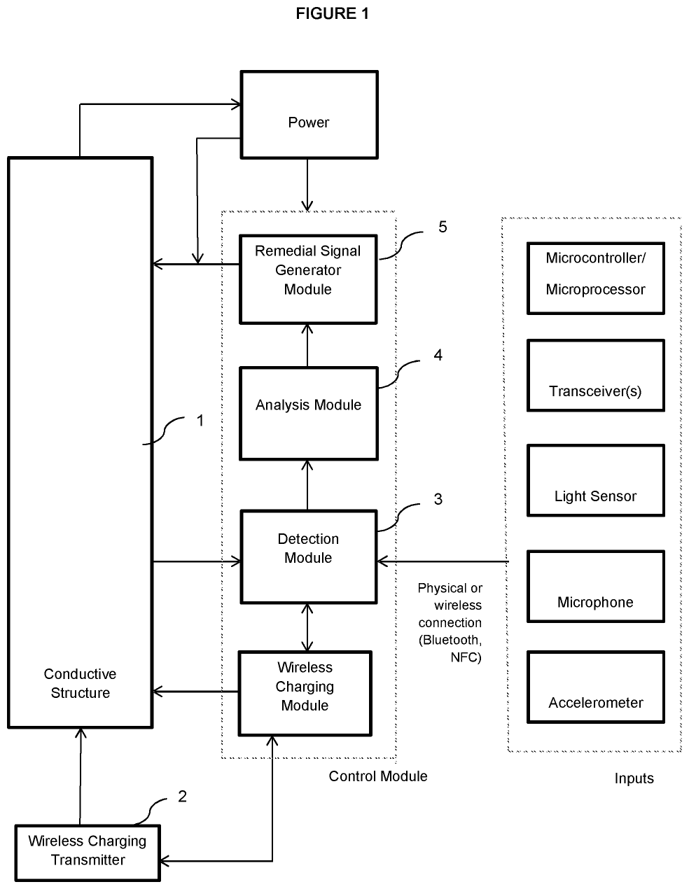

A schematic of a preferred device of the present invention is shown in FIG. 1.

In the design shown in FIG. 1 the conductive structure (1) enables inductive coupling with a wireless charging transmitter (2) to enable wireless power transfer to the module (3) to detect radiofrequency fields that may be emitted by the communication device and to create a remedial field as appropriate.

The detection module manages the flow of information from various inputs that is then used by the analysis module (4) to determine when to activate a remedial signal (5) and also the nature of the signal required. Inputs can include the radiation sensed by the conductive structure (an antenna); Bluetooth or near field communication (NFC) connection to an external communication device; information received directly from communication device microcontroller/transceivers; accelerometer; and other sensors in a communication device.

The control module typically comprises one or more microcontrollers which control the various different functions including signal detection, signal analysis, remedial signal generation and wireless charging and it allows for quick change of operation between different functions. Alternatively, a separate microcontroller can be used to control the wireless charging. In a preferred embodiment the control module deactivates the detection module when the wireless charging module is active.

The analysis module uses information gathered from the detection module to assess what remedial function to perform if any and it applies information gathered from the detection module to algorithms to determine how and when the remedial signal should be activated. In this way the remedial signal module controls the activation and parameters of the remedial signal, it can also control the power supplied to the conductive structure so that the remedial signal can be produced.

The wireless charging module charges the power source of the communication device typically the battery and controls the activation of wireless charging and preferably deactivates the detection module when wireless charging is active. The module can also provide wireless charging facilitated using the conductive structure and can communicate with the wireless charging transmitter to optimise wireless charging performance.

The signal detection module, the analysis module, the remedial signal activation module, and the wireless charging module may be implemented using a microcontroller and the preferred system comprises an electronic circuit that comprises a signal detection module, an analysis module and a remedial signal activation module, a wireless charging module and a multi-purpose conductive structure.

Accordingly, in a preferred embodiment the personal communication device contains the following modules:
Detection module
Control module
Analysis module
Remedial signal module
Wireless charging module
   And the various modules may contain the following features.

Detection Module

The detection module can comprise an Antenna which can be part of the conductive structure alternatively it may be a Bluetooth or near field communication (NFC) connection to an external communication device. The detection module can communicate with and receive information from a microcontroller/transceivers, a light sensor, an accelerometer, a microphone, other sensors in a communications device and a wireless charging station/transmitter. Information may be received via a wired or direct connection to the communication device.

Control Module

The control module may comprise one or more microcontrollers which control all the different functions: detection, analysis, remedial signal generation, wireless charging. The control modules allow for quick change of operation between different functions. Alternatively, a separate microcontroller can be used to control the wireless charging. In a preferred embodiment the control module deactivates the detection module when wireless charging module is active. The control module may comprise software or an app on the communication device.

Analysis Module

Uses information gathered from the detection module to assess if a remedial signal is required and determines the appropriate nature of the signal.

The Remedial Signal Module

Generates the remedial signal using the conductive structure.

Wireless Charging Module

Controls the wireless charging function facilitated using the conductive structure.

The conductive structure may be used as a wireless charging receiver and may be the same component used as the remedial signal generator such as a coil. In this way space can be saved within the mobile communication device allowing space for other components or the use of a larger battery. In a further embodiment the same conductive structure can be used as a detector to detect potentially harmful radiation.

In order to further reduce the size of the device the conductive structure can comprise multiple layers and different parts or sections of the conductive structure can be optimised for different purposes. As well as saving space the use of a multi-purpose conductive structure also allows the different functions to operate in the same location or within close proximity to one another within the device which is useful if wireless charging and remedial signal capabilities need to be located centrally within a communications device.

The conductive structure may be formed from conductive wire or conductive traces etched or printed on a printed circuit board or by printing conductive material onto plastic or a combination of these. In a preferred embodiment the structure is formed as a conductive trace on a printed circuit board which simplifies the design and reduces the number of required components and can reduce the complexity and cost of manufacturing the device and integrating the device into a communications device. In one embodiment the conductive structure can be a combination of a printed circuit board trace or traces and one or more wires or coils.

The conductive structure is designed to optimise the coupling between the wireless charging transmitter and receiver to ensure quick and efficient wireless power transfer and simultaneously designed to efficiently produce a remedial signal of the required strength. In a further embodiment the conductive structure and wireless charging module are designed to have a specific q factor and are also designed to comply with specific wireless charging standards such as the Qi standard. The performance of the conductive structure can be improved by providing a conductive backing such as a ferrite strip or plate to optimise power transfer between the wireless transceiver and receiver.

In our preferred embodiment a micro-controller operates the entire system controlling each of the different functions and it also operates a timer to check periodically if the potentially harmful signal is present. The micro-controller may also be programmed to recognise the type of radio frequency signal that is being detected and to monitor the battery power of the communication device and the remaining battery life.

In a preferred embodiment, the microcontroller operates both the remedial device and also the wireless charging function. In a further embodiment the wireless charging module is controlled by a separate microcontroller.

In a further embodiment the wireless charging mechanism comprises a SafeWave wireless charging tags or stuck into the power port of a phone, or stuck on the back of the phone and/or fit under a phone case. The printed circuit board (PCB) is preferably at the top of the device and includes a trace coil that creates the remedial field and a detector loop which detects the radiation emitted from the phone. The PCB also contains microcontrollers: to control the detection, analysis and remedial field, and one to control the wireless charging. The wireless charging coil preferably sits below the PCB and typically consists of a coil of wire on a ferrite backing which enhances the efficiency of the wireless charging. So the conductive structure will consist of a trace coil in the PCB to produce the remedial field, a trace detector loop in the PCB to detect the radiation from the phone, and a wire coil as the wireless charging receiver.

Alternatively, the Phone case can plug into the phone and draw power directly from the iPhone. The case will include a wire coil to produce the remedial field near the top of the case which is controlled from a microcontroller on an embedded PCB at the bottom of the case. The case will be designed to allow the existing wireless charging coil built into the Phones to operate without interference. The case will gather information directly from the phone. The control module will either be run from the microcontroller on the PCB or using a software on the Phone in the form of an app (or a combination of both). The Phone case will have a passthrough connector which allows you to charge your phone using a cable through the case. The conductive structure will be two wire coils: one in the case used to create the remedial field and the existing coil built into the handset for wireless charging.

The device can be integrated directly into a telephone handset or other types of communications device or integrated into a case for a communications device or integrated into a thin card device that can be placed next to a communications device.

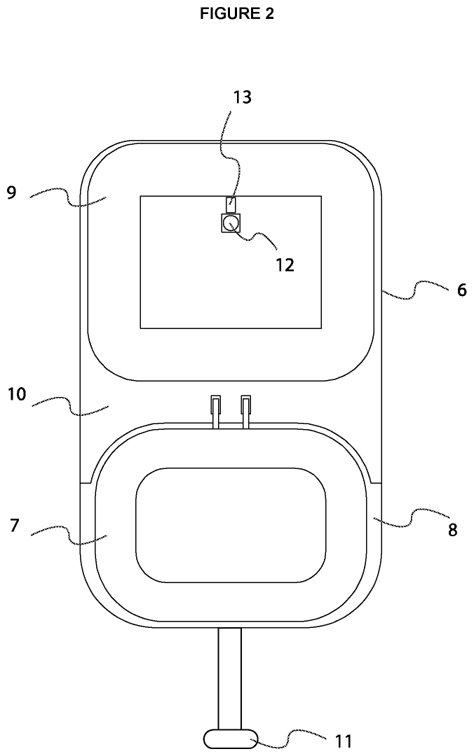

FIG. 2 illustrates a wireless charging assembly (6) which can be in the form of a card and can be applied to either a mobile telephone or to a case for the telephone. The assembly is provided with a wireless charging coil (7) having a ferrite backing (8) and also a trace coil (9) on a printed circuit board (10) for generation of a remedial field when required by the detection module (not shown). The assembly (6) can receive power and information from the communication device through a connector (11) and can include a button (12) for activating or controlling the device and a light (13) to indicate different modes or functions.

1. A communication device that emits radio frequency signals comprising:
a module to determine when the radio frequency signals may be present and are potentially harmful, and to activate a remedial signal generator if the radio frequency signals are considered to be potentially harmful; and
wherein a power to operate the module is from a power source that can be charged using wireless charging techniques.

2. The communication device according to claim 1, wherein a wireless charging of the power source is achieved by provision of a conductive structure that acts as a wireless charging receiver for receipt of transmission from a remote wireless charging transmitter.

3. The communication device according to claim 1, wherein the communication device emits a potentially harmful radiation; and
wherein effects of the potentially harmful radiation are reduced or eliminated through a superposition of a remedial signal and this remedial signal is generated using the power source that can be charged using wireless charging.

4. The communication device according to claim 1, comprising:
a) a portable telephone; and
b) a module to include a receiver of wireless charging provided within a handset or a case of the portable telephone.

5. The communication device according to claim 1 comprising:
a) a detection module that receives information used to determine when a radiation which is potentially harmful may be present;
b) an analysis module that analyzes the received information to determine if it is potentially harmful and also a nature of the radiation in order to determine a type, a strength, and/or a duration of a remedial signal that is appropriate for the radiation that has been sensed.

6. The communication device according to claim 5, containing a control module;
and wherein the control module also contains a remedial signal generator module which can be activated by the analysis module to generate the remedial signal which is appropriate.

7. The communication device according to claim 1, wherein all functions are provided by a single microprocessor which is powered by the power source that can be charged wirelessly.

8. The communication device according to claim 7, wherein the power source comprises a battery.

9. The communication device according to claim 6, wherein the detection module manages a flow of the information from various inputs that is then used by the analysis module to determine when to activate the remedial signal and also a nature of the remedial signal required.

10. The communication device according to claim 6, wherein the control module deactivates the detection module when a wireless charging module is active.

11. The communication device according to claim 5, wherein the analysis module uses information gathered from the detection module to assess what remedial function to perform, if any, and the analysis module applies information gathered from the detection module to algorithms to determine how and when the remedial signal should be activated.

12. The communication device according to claim 1, wherein a wireless charging module charges the power source of the communication device, controls activation of wireless charging, and deactivates a detection module when the wireless charging is active.

13. The communication device according to claim 1, wherein a detection module, an analysis module, a remedial signal generator module, and a wireless charging module are implemented using one or more microcontrollers or microprocessors.

14-16. (canceled)

17. The communication device according to claim 2, wherein the conductive structure is used to create a remedial signal.

18. The communication device according claim 2, wherein the conductive structures is used to detect a potentially harmful radiation.

19. The communication device according to claim 2, wherein the conductive structure is formed of multiple layers.

20. A communication device that emits radio frequency signals comprising:
a) a control module to determine when the radio frequency signals may be present and are potentially harmful, and to activate a remedial signal generator module if the radio frequency signals are considered to be potentially harmful, the control module including:
i) a detection module that receives information used to determine when a radiation which is potentially harmful may be present;
ii) an analysis module that analyzes the received information to determined if it is potentially harmful and also a nature of the radiation in order to determine a type, a strength, and/or a duration of a remedial signal that is appropriate for the radiation that has been sensed;
iii) a remedial signal generator module which can be activated by the analysis module to generate the remedial signal which is appropriate;
b) a conductive structure that acts as a wireless charging receiver for receipt of transmission from a wireless charging transmitter;
wherein a power to operate the control module is from a power source that can be charged using a wireless charging; and
wherein the wireless charging of the power source is achieved by the conductive structure.

21. The communication device according to claim 20, wherein the detection module manages a flow of the information from various inputs that is then used by the analysis module to determine when to activate the remedial signal and also a nature of the remedial signal required.

22. The communication device according to claim 20, wherein the control module includes a wireless charging module; and
wherein the control module deactivates the detection module when a wireless charging module is active.

23. The communication device according to claim 20, wherein the conductive structure is used to create the remedial signal.

24. The communication device according to claim 20, wherein all functions are provided by a single microprocessor which is powered by the power source that can be charged wirelessly.
